(19) United States
(12) Patent Application Publication
(10) Pub. No.: US 2014/0364821 A1
(43) Pub. Date: Dec. 11, 2014

(54) SYSTEM AND METHOD FOR DRAINING FLUIDS

(71) Applicant: Kevin Gibbons, Orchard Park, NY (US)

(72) Inventor: Kevin Gibbons, Orchard Park, NY (US)

(73) Assignee: The Research Foundation for The State University of New York, Amherst, NY (US)

(21) Appl. No.: 14/001,513

(22) PCT Filed: Feb. 27, 2012
(86) PCT No.: PCT/US12/26785

§ 371 Date: Jun. 4, 2014

Related U.S. Application Data
(60) Provisional application No. 61/446,612, filed on Feb. 25, 2011.

(51) Int. Cl. A61M 1/00

(52) U.S. Cl. CPC A61M 1/0035; A61M 1/0084; A61M 2202/0464; A61M 2210/0693
USPC 604/319

(57) ABSTRACT

A system and/or a method of evacuating patient fluid from a subdural space of a patient. The system includes a flexible subdural drain having a plurality of apertures and a port in fluid communication with a valve. A reservoir containing a cleansing fluid is in fluid communication with the valve. An outlet in fluid communication with the valve. In a flush state, the valve is configured to establish fluid communication between the reservoir and the flexible subdural drain for evacuating the cleansing fluid from the reservoir. In a priming state, the valve is configured to close the fluid communication between the reservoir and the flexible subdural drain. In a drain state, the valve is configured to establish fluid communication between the flexible subdural drain and the outlet for evacuating the patient fluid via a siphoning effect through the outlet.

### CROSS-REFERENCE TO RELATED APPLICATION

This application claims the benefit of priority to U.S. provisional patent application Ser. No. 61/446,612, filed on Feb. 25, 2011, the disclosure of which is incorporated herein by reference.

### FIELD OF THE INVENTION

The present invention relates generally to draining fluids from an individual, and in particular, to draining fluids from the subdural space of an individual.

### BACKGROUND OF THE INVENTION

Chronic subdural hematomas are a common neurosurgical condition, seen with increasing prevalence due to our aging population and more widespread indication for and use of anticoagulant and antiplatelet medications. Additionally, increased numbers of patients are presenting with bilateral subdural collections, posing certain problems in operative management.

Recent studies have demonstrated that burr hole drainage is preferred over the alternatives of twist drill craniotomy and full craniotomy. Additionally, recent studies have demonstrated better outcomes in patients in whom subdural drains are left in place postoperatively versus no drain. However, drain placement is associated with certain risks of serious injury, including cerebral contusion, acute subdural and intraparenchymal hemorrhage, and intraparenchymal placement. Subdural drains are often ineffective, with significant residual air and subdural fluid (both residual hematoma and saline irrigant placed at surgery to washout hematoma contents including native fibrinolytic substances) seen on postoperative imaging. Subdural air in and of itself is often harmful, as well as a major predictor of subdural hematoma recurrence. Finally, studies have demonstrated that the greater the volume of drainage after surgery, and the less air seen on postoperative scans, the better patients do and the lower the rate of recurrence.

General neurosurgical-use drains for treating subdural hematomas were not generally designed for the purpose of treating chronic subdural hematomas. One commercially available system for subdurals does not facilitate intraoperative drainage and irrigation.

Therefore, there is a need for drains that can be effectively implemented for treating subdural hematomas, particularly chronic subdural hematomas, and devices, systems, and methods for inserting and implementing drainage of the subdural space.

### BRIEF SUMMARY OF THE INVENTION

A system for evacuating patient fluid from a subdural space of a patient according to the present invention comprises a valve, a flexible subdural drain, a reservoir, and an outlet. The valve can have selectable states, including a flush state, a priming state, and a drain state. The flexible subdural drain has a plurality of apertures and a port. The port is in fluid communication with the valve. The reservoir contains a cleansing fluid and is in fluid communication with the valve. The outlet is in fluid communication with the valve. In the flush state, the valve is configured to establish fluid communication between the reservoir and the flexible subdural drain for evacuating the cleansing fluid from the reservoir. In the priming state, the valve is configured to close the fluid communication between the reservoir and the flexible subdural drain. In the drain state, the valve is configured to establish fluid communication between the flexible subdural drain and the outlet for evacuating the patient fluid via a siphoning effect through the outlet.

According to another embodiment of the invention, a method of evacuating patient fluid from a subdural space of a patient is provided. The method includes placing a flexible subdural drain having a plurality of apertures into the subdural space of the patient. A valve is selectively operated to establish fluid communication between a reservoir containing cleansing fluid and the flexible subdural drain. Cleansing fluid from the reservoir, is evacuated through the flexible subdural drain, and into the subdural space of the patient. The valve is selectively operated to close the fluid communication between the reservoir and the flexible subdural drain. The valve is selectively operated to establish fluid communication between the flexible subdural drain and an outlet. The patient fluid is siphoned from the subdural space of the patient through the outlet.

### DESCRIPTION OF THE DRAWINGS

For a fuller understanding of the nature and objects of the invention, reference should be made to the following detailed description taken in conjunction with the accompanying drawings, in which:

FIG. 1 depicts a system according to an embodiment of the invention;

FIG. 2A is a top view of an embodiment of a flexible subdural drain;

FIG. 2B is a side view of the flexible subdural drain of FIG. 2A;

FIG. 3A depicts a system according to another embodiment of the invention;

FIG. 3B depicts an alternate reservoir configuration according to an embodiment of the present invention;

FIG. 4A is cross-sectional view of a guide tool;

FIG. 4B is a side-view of a guide tool being used to insert a subdural drain of the present invention;

FIG. 5A depicts various positions of a patient's head on a prior art headrest;

FIG. 5B depicts various positions of a patient's head on a headrest according to the present invention; and

FIG. 6 depicts a method according to the present invention.

### DETAILED DESCRIPTION OF THE INVENTION

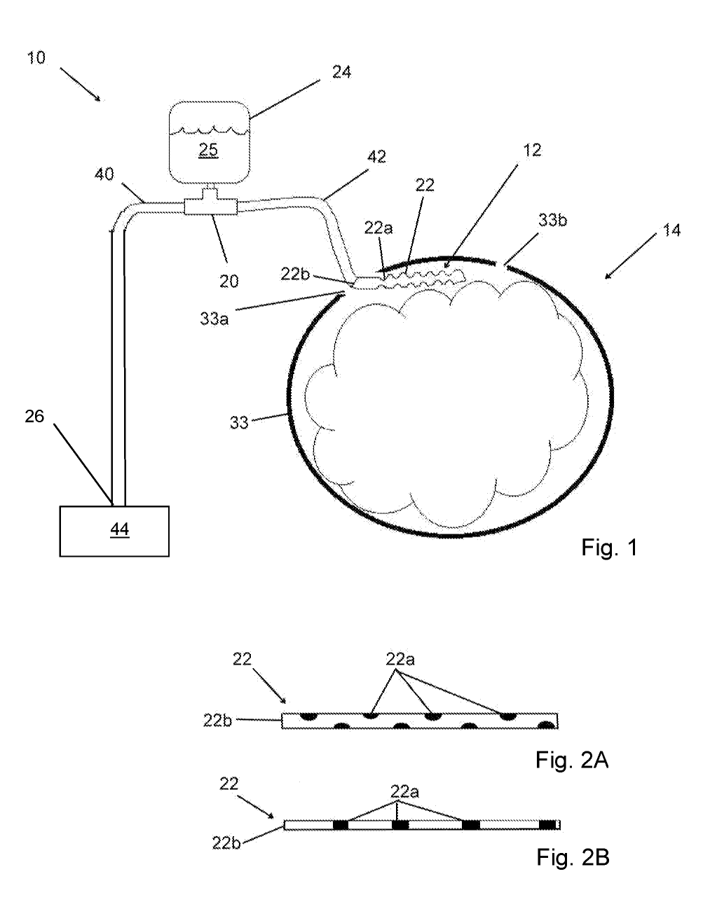

FIG. 1 depicts a system 10 for evacuating fluid from a subdural space 12 of an individual 14. As used herein, “fluid from the subdural space” is a broad term that can include at least one or more of the following: air, cleansing fluid, spinal fluid, blood, hematoma, hygroma, solid clot, unclotted arterial, or any other fluid that may be found during draining the subdural space from a patient. The system 10 generally comprises a valve 20, a flexible subdural drain 22, a reservoir 24, and an outlet 26. The reservoir 24 contains a cleansing fluid 25, for example, an isothermic saline solution, that can be used to irrigate the subdural space 12. The cleansing fluid 25 may be used to promote removal of broken down hematoma, solid clot, unclotted arterial, and venous blood. The reservoir 24 may be embodied as a closed bag, an open bag or reservoir, a syringe, or any container suitable for containing a fluid. In one embodiment, the reservoir 24 has at least a 300 cc capacity. For example, a large volume bag, capable of holding 500 cc may be used.

The valve 20 can have three branch openings on the periphery thereof and a flow passage switching portion. Each branch opening provides respective fluid communication to the flexible subdural drain 22, the reservoir 24, and the outlet 26. The valve 20 can be selectively operated to adjust the flow passage switching portion, such that fluid communication is adjusted between the various branch openings. In this way, the valve 20 has at least three states: a flush state, a priming state, and a drain state. In the flush state, the valve 20 is configured to establish fluid communication between the reservoir 24 and the port 22b of the flexible subdural drain 22. In the drain state, the valve 20 is configured to establish fluid communication between the port 22b of the flexible subdural drain 22 and the outlet 26. In the priming state, the valve 20 is configured to close the fluid communication between the reservoir 24 and the flexible subdural drain 22. The valve 20 may also have a closed state in which all branch openings are closed. The valve 20 may be embodied as a conventional three-way stopcock, known in the medical field. Each of the branch openings may be individually closeable. In one embodiment, the valve 20 can only allow, at a maximum, fluid communication between two of the three respective branch openings at a time.

FIG. 2A is a top view of a flexible subdural drain 22, and FIG. 2B is a side view of the flexible subdural drain 22, according to the present invention. The flexible subdural drain 22 has a plurality of apertures 22a and a port 22b in fluid communication with the valve 20. In one embodiment, the flexible subdural drain 22 is a 4 mm flat silicone drain, such as a flat TLS® drain manufactured by POREX, STRYKER. The TLS® drain, which is currently used for ear, nose, and throat (“ENT”) and plastic surgical procedures, has a planar top, a planar bottom, and rounded sides. Apertures 22a are located on the rounded sides and on the tip of TLS® drains. TLS® drains are smaller in length and width, and are softer and more flexible than drains traditionally used to drain the subdural space 12 of a patient 14, such as large bore ventricular catheters and Jackson Pratt drains. TLS® drains also have larger apertures than other drains traditionally used to drain the subdural space.

A section of distal tubing 40, which can be approximately 4-6 feet in length, can be used to connect the valve 20 to the outlet 26. A collector 44 for collecting fluid may be used to collect fluid, including fluid from the subdural space 12, from the outlet 26. The collector 44 may be large enough to avoid having to replace during the drainage process and can conveniently be placed on the floor. Typically, a collector 44 having at least 500 cc capacity is sufficient. Non-limiting examples of collectors 44 include open or closed bags, trays, tubes, and drains.

In a surgical procedure using a system 10 according to the present invention, the subdural space 12 is exposed through two bun holes 33a, 33b each having a diameter of approximately 8-20 mm, and opening of the dura. The subdural space 12 is irrigated with isothermic saline to promote removal of broken down hematoma (“motor oil”), solid clot, and unclotted arterial and venous blood. The enlarged subdural space 12 rarely collapses immediately by reexpanded brain parenchyma. Upon closure, there can be a collection of air, saline, and residual that is often as large or larger than the original clot. This collection creates a need for drainage postoperatively.

In one embodiment, the flexible subdural drain 22 is inserted through the posterior bun hole 33a, directed posteriorly in the subdural space 12, and tunneled out through the anterior burr hole 33b. The valve 20 is selectively operated into the flush state such that fluid communication is established between the flexible subdural drain 22 and the reservoir 24. By positioning the reservoir 24 above the flexible subdural drain 22, gravity will cause the cleansing fluid 25 to flow out of the reservoir 24, through the apertures 22a of the subdural drain 22, and into the subdural space 12. In one embodiment, the reservoir 24 is positioned approximately 10 cm above the patient's head 33 to provide a suitable flow of cleansing fluid 25. The reservoir 24 may be an open reservoir, which can be repeatedly refilled while the burr holes 33a, 33b are closed in an air and watertight fashion. By passively gravity feeding the cleansing fluid 25 into the subdural space, unnecessary mass effect or intracranial pressure is avoided. During a surgical procedure using a system 10 according to the present invention, the subdural space 12 is under constant passive irrigation via the reservoir 24 while the posterior burr hole 33b is closed first, and the anterior burr hole 33a second. In one embodiment, the patient's head 33 is positioned such that the anterior burr hole 33a is at the high point of the head 33. In this fashion, air is allowed to escape the head 33 during closure of the anterior burr hole 33a by the constant irrigation from cleansing fluid 25 flow out of the subdural drain 22. Once irrigation is concluded, the valve 20 can be selectively operated into the priming state, such that fluid communication between the reservoir 24 and the flexible subdural drain 22 is closed.

The system 10 may include a priming source for producing a negative pressure to induce a siphoning effect. The siphoning effect can cause fluid to be sucked away from the subdural space 12 postoperatively. In one embodiment, the priming source is the reservoir 24. To use the reservoir 24 as the priming source, the valve 20 is selectively operated into the priming state such that fluid communication between the reservoir 24 and the flexible drain 22 is closed. Fluid communication between the reservoir 24 and the outlet 26 can then be established to allow fluid 25 from the reservoir 24 to flow through the distal tubing 40 toward the outlet 26. This fluid flow through the distal tubing 40 creates negative pressure on the system 10 for producing the siphoning effect. The valve 20 can next be placed in a drain state, such that the valve 20 is configured to establish fluid communication between the flexible drain 22 and the outlet 26. During the drain state, the valve 20 can close fluid communication to the reservoir 24. The negative pressure from the fluid flow through the distal tubing 40 produces the siphoning effect, which sucks fluid from the subdural space 12 in from the flexible subdural drain 22, and out through the outlet 26. Alternatively, the priming source can be a pump, an actuator, a fluid reservoir, or any other suitable pressure source. For example, a secondary reservoir can be included in the system 10 to induce the siphoning effect. It has been found that approximately 3-5 cc of fluid flow through the distal tubing 40, to an outlet 26 approximately at ground level, is sufficient to produce an adequate siphon.

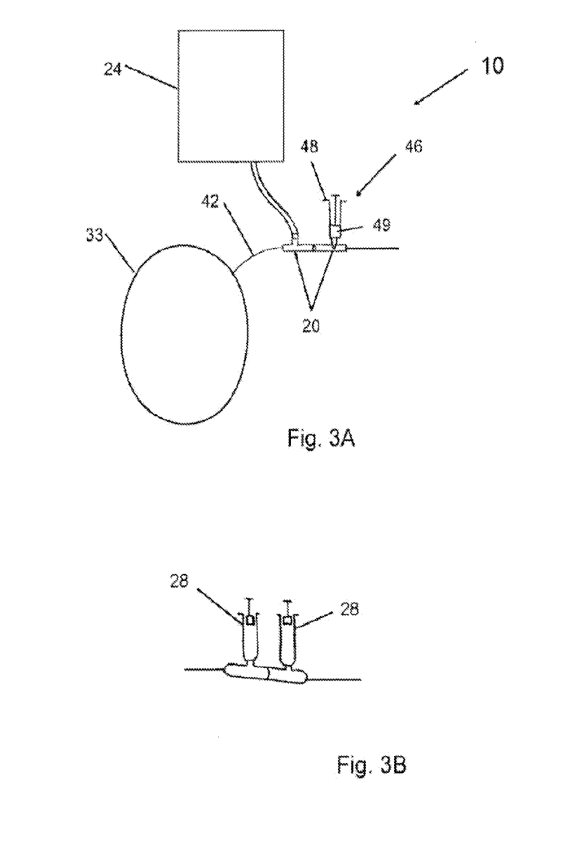

The siphoning effect may also, advantageously, draw in subdural air. Because the air can cause a break the siphoning effect, the siphoning effect will need to be reestablished. In an embodiment depicted in FIG. 3A, the system 10 includes a priming system 46, including a priming reservoir 48 containing a liquid 49 configured to create a negative pressure for reestablishing the siphoning effect. However, the priming system 46 can act as the priming source described above, and therefore be used to produce a siphoning effect at any time. Typically, flushing 4 ml of fluid 49 from the priming reservoir 48 is sufficient to reestablish subdural fluid flow from the flexible subdural drain 22. If such a flush does not reestablish subdural fluid flow, there may be a proximal clot or obstruction. In that case, a proximal clot or obstruction can be cleared by flushing the system proximally with less than 5 cc of fluid. For example, the valve 20 can be selectively operated to establish fluid communication between the reservoir 24 and the subdural drain 22 or the priming reservoir and the subdural drain 22, for flushing. A flow of fluid, e.g. fluid 25, 49, can be used to flush the proximal tubing 42 and/or the subdural drain 22. Any drain manipulation, re-priming, or flushing can be done without any break or opening of the system 10 to maintain a sterile surgical environment. Maximal drainage occurs over 12-24 hours, and the drain 22 is removed by 24 hours post-operation in the vast majority of cases.

In one embodiment of the present invention, the reservoirs 24, 48 are controlled volume syringe injectors. For example, a controlled volume syringe with a large reservoir may be utilized, such as a syringe having a 120 cc volume configured to inject at a maximum 5 cc of fluid per injection. Because the re-priming is routinely done by physicians and nurses in the Intensive Care Unit postoperatively, controlled volume syringe injectors facilitate simple, safe, effective, and standardized re-priming of the system. Further, controlled volume syringe injectors make it difficult for a patient or visitor to inadvertently or intentionally inject a large volume of saline proximally into the head.

In some cases, extra-large reservoirs 24 or a series of reservoirs 24 may be used in the system 10 for draining extremely large clots/subdural spaces 12. It has been found that approximately one fifth of patients require more than 10-12 re-primings (at approximately 3-5 cc each). For example, the system 10 may include a reservoir 24 larger than 60 cc, in conjunction with an additional 60 cc reservoir 24. Each additional reservoir 24 should be in fluid communication with the system 10, and have a respective valve 20.

In one embodiment, a large volume reservoir bag, such as a 500 cc bag of saline, and an additional large 60 cc syringe are attached to the system 10. The large volume bag may be attached to a valve 20 that is in series with a second valve 20 to which a 5 cc syringe is attached. The 5 cc syringe can be loaded for each siphon re-priming by closing the valves 20 to the patient and the outlet 26, and drawing the desired 5 cc of fluid 25 from the large saline bag. In another embodiment, shown in FIG. 3B, two 60 cc syringes 28 are arranged in parallel for use as reservoirs. This allows the system 10 to be easily broken, sterilely, to replace one or more of the syringes 28. For example, an empty 60 cc syringe 28 can be replaced with a 60 cc syringe 28 filled with fluid. It should be understood that the syringe 28 can be embodied as any reservoir, can be various sizes, and does not need to be 60 cc.

Drainage can be ceased by placing the valve 20 in the closed state, such that there is no fluid communication between branch openings of the valve. The subdural drain 22 may then be removed from the subdural space 12. The drain 12 may be removed when fluid from the subdural space 12 can no longer be drained, or when diagnostic imaging shows that a sufficient amount of fluid from the subdural space 12 has been removed.

Current devices used to facilitate directional subdural drain placement are inadequate. They are small metal instruments, typically a dural separator, penfield #3, or a Woodson or dental instrument. Their sole function is to prevent placement into the brain parenchyma. The top part, toward the skull, on the concave side of the instrument, is itself convex, preventing channeling of the drain in a particular direction.

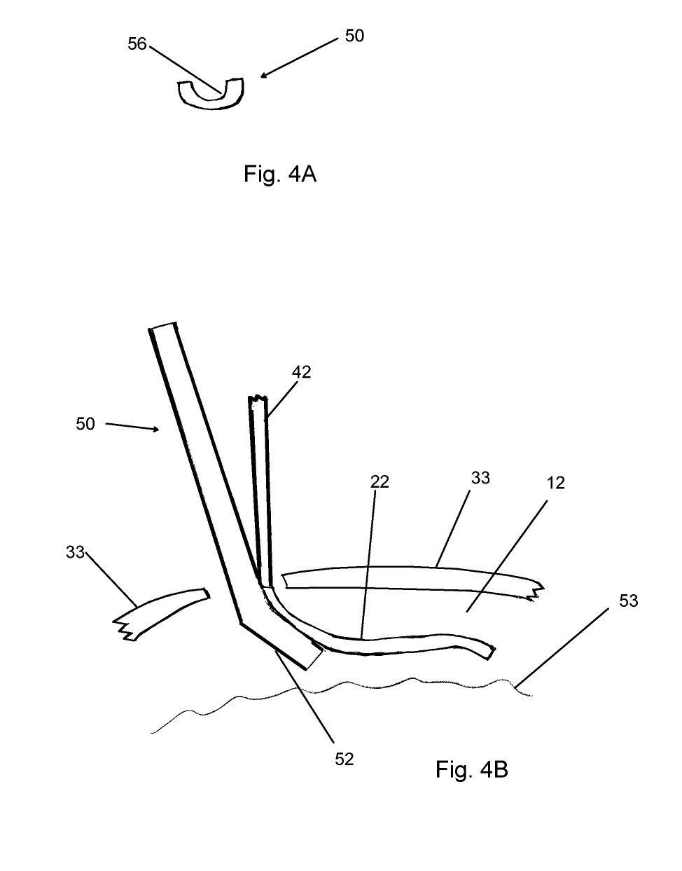

FIGS. 4A and 4B depict a guide tool 50 that may be used in conjunction with the present invention. The guide tool 50 may be used to direct the subdural drain 22 into the subdural space 12 (typically posteriorly). The guide tool 50 can have a bottom 52 that is angled, curved, or convex relative to the length 54 of the guide tool 50, such that the bottom 52 urges the subdural drain 22 away from the brain 53 during insertion. As shown in the cross-sectional view of FIG. 4B, the guide tool 50 can have a groove 56 sized to receive the flexible subdural drain 22. The groove 56 can be disposed along the entire length 54, or a portion thereof, of the guide tool 50 and the bottom 52. In one embodiment, the groove 56 is 5 mm wide. The length of the guide tool 50 can be both straight and/or curved, depending on whether the burr hole 33a, 33b is conventional (saucerized edge) or tangential. The guide tool 50 can also include a side angle grip design to ease placement of the subdural drain 22.

The number of patients with bilateral chronic subdural hematomas, requiring bilateral drainage is increasing. However, there are certain difficulties in surgical set up and performance of such a procedure. The preferred anterior burr hole for most convexity subdural is mid frontal, at or behind the hairline, 3-8 cm from midline. Patients are rotated such that the anterior burr hole is the highest point on the head to minimize residual air after drainage. Patients who undergo bilateral drainage procedures with the head in the neutral position often have very radiographic and clinically significant pneumocephalus postop, putting them at risk for significant neurologic compromise, acute subdural hemorrhage formation from vein tearing, seizures, hyponatremia, and recurrent chronic subdural hematomas. Two separate procedures, with complete closure, repositioning and redraping, extends surgery and anesthesia time.

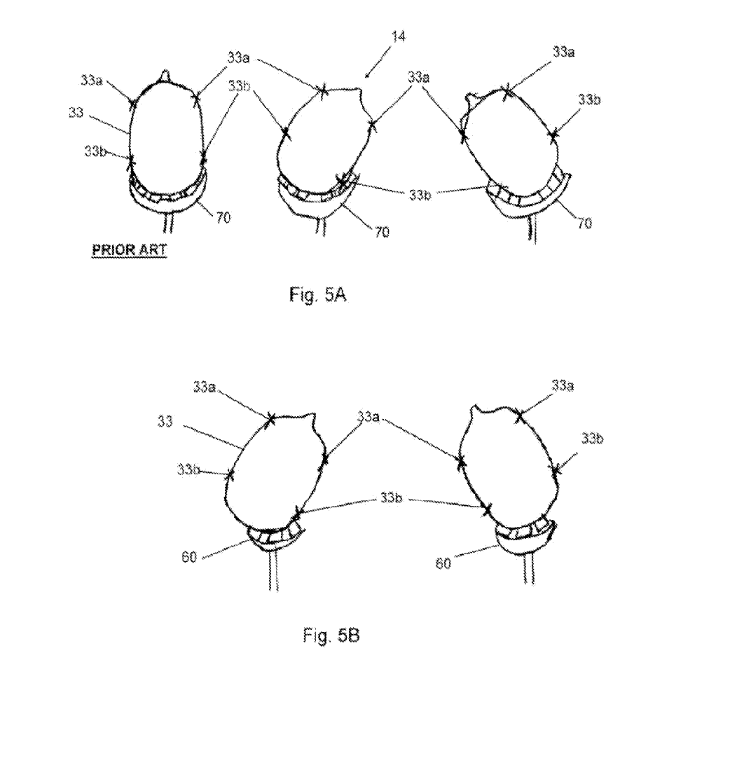

The present invention may also include a headrest 60, designed for bilateral procedures, which may be used in accordance with the present invention. FIG. 5A depicts a prior art headrest 70 with a patient's 14 head 33 in various positions. The prior art headrest 70 is designed to support the dependent third of the circumference or arc of the head 33. However, when the patient 14 is rotated such that an anterior burr hole 33a is at its highest point, the headrest 70 covers the downside (the side of the head that is not actively being worked on) posterior burr hole 33b. As shown in FIG. 5B, a headrest 60 according to the present invention may be contoured to the shape of the lateral occiput, but be sized such that the headrest does not reach the parietal/temporal area of the downside. In this manner, adequate support of the head 33 is facilitated by the headrest, without blocking the posterior burr holes 33b of each side the head 33. The headrest 60 may be mounted to a traditional Mayfield® headrest, or be on a slide that is placed on the operating room table under the cushion bearing the weight of the patient's torso.

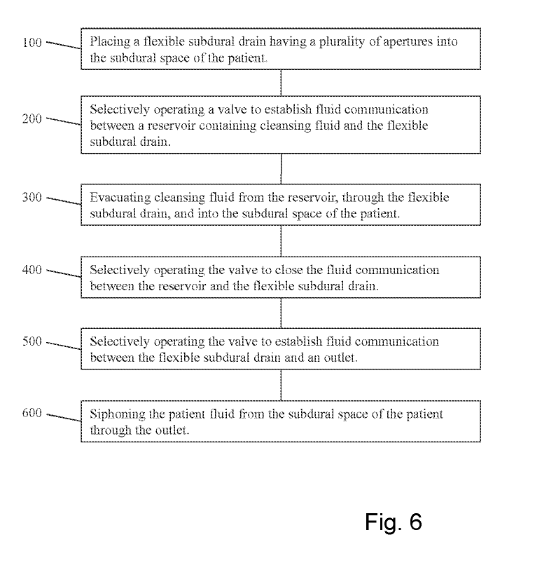

The present invention may be embodied as a method of evacuating fluid from a subdural space of a patient. As shown in FIG. 6, the method comprises placing 100 a flexible subdural drain having a plurality of apertures into the subdural space of the patient.

The flexible subdural drain can be placed into the subdural space with a guide tool, the guide tool including an angled bottom and a groove sized to receive the flexible subdural drain, the groove being disposed along a length of the guide tool and the angled bottom. A valve is selectively operated 200 to establish fluid communication between a reservoir containing cleansing fluid and the flexible subdural drain. Cleansing fluid from the reservoir is evacuated 300, through the flexible subdural drain, and into the subdural space of the patient. The valve is selectively operated 400 to close the fluid communication between the reservoir and the flexible subdural drain. The valve is selectively operated 500 to establish fluid communication between the flexible subdural drain and an outlet. The fluid is siphoned 600 from the subdural space of the patient through the outlet. In a preferred embodiment, the fluid is siphoned through the outlet into a collector, such as a fluid collection bag.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A system for evacuating patient fluid from a subdural space of a patient, comprising:
a valve having selectable states, including a flush state, a priming state, and a drain state;
a flexible subdural drain having a plurality of apertures and a port, the port in fluid communication with the valve;
a reservoir containing a cleansing fluid, the reservoir in fluid communication with the valve;
an outlet in fluid communication with the valve;
wherein in the flush state, the valve is configured to establish fluid communication between the reservoir and the flexible subdural drain for evacuating the cleansing fluid from the reservoir;
wherein in the priming state, the valve is configured to close the fluid communication between the reservoir and the flexible subdural drain;
wherein in the drain state, the valve is configured to establish fluid communication between the flexible subdural drain and the outlet for evacuating the patient fluid via a siphoning effect through the outlet.

2. The system of claim 1, further comprising a collector in fluid communication with the outlet for collecting fluid.

3. The system of claim 2, wherein the collector is a fluid collection bag.

4. The system of claim 1, wherein the flexible subdural drain has a planar top, a planar bottom, and rounded sides, wherein the plurality of apertures are located on the rounded sides.

5. The system of claim 4, wherein the flexible subdural drain is a 4 mm flat silicone drain.

6. The system of claim 1, further comprising a guide tool for placing the flexible subdural drain into the subdural space.

7. The system of claim 6, wherein the guide tool includes an angled bottom and a groove sized to receive the flexible subdural drain, the groove being disposed along a length of the guide tool and the angled bottom.

8. The system of claim 7, wherein the angled bottom is curved in a convex direction, away from the length of the guide tool.

9. The system of claim 1, wherein the cleansing fluid from the reservoir creates a negative pressure to produce the siphoning effect.

10. The system of claim 1, wherein a pump creates a negative pressure to produce the siphoning effect.

11. The system of claim 1, wherein between approximately 3-5 cc of saline creates a negative pressure to produce the siphoning effect.

12. The system of claim 1, further comprising a priming system, including a priming reservoir containing a liquid configured to create a negative pressure for producing the siphoning effect.

13. The system of claim 10, wherein the priming reservoir is a syringe.

14. The system of claim 12, wherein the priming system is configured to produce multiple siphoning effects for reestablishing the negative pressure after a loss of the siphoning effect.

15. A method of evacuating patient fluid from a subdural space of a patient, comprising:
placing a flexible subdural drain having a plurality of apertures into the subdural space of the patient;
selectively operating a valve to establish fluid communication between a reservoir containing cleansing fluid and the flexible subdural drain;
evacuating cleansing fluid from the reservoir, through the flexible subdural drain, and into the subdural space of the patient;
selectively operating the valve to close the fluid communication between the reservoir and the flexible subdural drain;
selectively operating the valve to establish fluid communication between the flexible subdural drain and an outlet; and
siphoning the patient fluid from the subdural space of the patient through the outlet.

16. The method of claim 15, wherein the patient fluid is siphoned through the outlet into a collector.

17. The method of claim 16, wherein the collector is a fluid collection bag.

18. The method of claim 15, wherein the flexible subdural drain is placed into the subdural space with a guide tool, the guide tool including an angled bottom and a groove sized to receive the flexible subdural drain, the groove being disposed along a length of the guide tool and the angled bottom.

19. The method of claim 15, wherein the siphon is produced by a priming system, including a priming reservoir containing a liquid.

20. The method of claim 19, further comprising establishing at least one subsequent siphon to evacuate the patient fluid from the subdural space after interruption of the siphon.
